(19) United States
(12) Plant Patent Application Publication
(10) Pub. No.: US 2002/0059671 P1
(43) Pub. Date: May 16, 2002

(54) Lily plant named 'genova'

(76) Inventor: Cees A. van der Voort, Jn Katwijk (NL)

Correspondence Address: Foley & Lardner, Washington Harbour, 3000 K Street NW, Suit 500, Washington, DC, 20007-5109, US

(21) Appl. No.: 09/521,606

(22) Filed: Mar. 9, 2000

(51) Int. Cl. A01H 5/00

(52) U.S. Cl. PLT/315

(57) ABSTRACT

A new and distinct cultivar of Lily plant named ‘Genova’ characterized by its erected, light-pink flowers, contrasting orange pollen and markings on the inner side of the throat, vigorous growth and long storage capabilities.

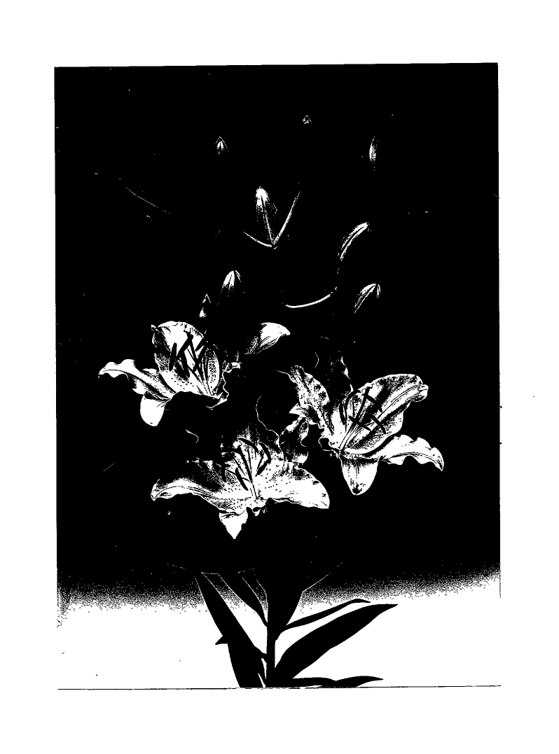

### BACKGROUND OF THE INVENTION

The present invention comprises a new and distinct cultivar of lily plant, botanically known as Lilium L., and hereinafter referred to by the cultivar name ‘Genova’.

‘Genova’ is a product of a planned breeding program which had the objective of creating new lily cultivars having many large erected flowers and vigorous growth.

‘Genova’ was originated from a hybridization made by the inventor in a controlled breeding program in Rijnsburg, the Netherlands in 1990. The male and female parents were unnamed seedlings of Lilium L. oriental hybrids. Both parents are proprietary cultivars used in the breeding program.

‘Genova’ was discovered and selected as one flowering plant within the progeny of the stated cross by the inventor, Cees A. van der Voort, in the months of May/June 1990 in a controlled environment in Rijnsburg, the Netherlands.

The first act of asexual reproduction of ‘Genova’ was accomplished when scales were taken from the initial selection in October 1992 in a controlled environment in Rijnsburg, the Netherlands by, or under the supervision of, Cees A. van der Voort. Horticultural examination of selected units initiated in May, 1992 has demonstrated that the combination of characteristics as herein disclosed for ‘Genova’ are firmly fixed and are retained through successive generations of asexual reproduction.

### BRIEF DESCRIPTION OF THE INVENTION

The following traits have been repeatedly observed and are determined to be basic characteristics of ‘Genova’ which in combination distinguish this lily as a new and distinct cultivar:

1. large, erect flowers;

2. tepals with curled edges;

3. soft pink color with contrasting orange pollen; and

4. vigorous growth habit.

‘Genova’ has not been observed under all possible environmental conditions. The phenotype of the new cultivar may vary significantly with variations in environment such as temperature, light intensity, and daylength without any change in genotype. The following observations, measurements, and comparisons describe plants grown in the experimental research station CPRO-DLO, Wageningen, the Netherlands under conditions which approximate those generally used in commercial practice.

Of the many commercial cultivars known to the present inventor, no other cultivars are similar in comparison to ‘Genova’.

### BRIEF DESCRIPTION OF THE DRAWING

The accompanying color photographic illustration shows typical plant and flower characteristics of ‘Genova’, with colors being as true as possible with illustrations of this type.

### DETAILED BOTANICAL DESCRIPTION

In the following description, color references are made to The Royal Horticultural Society (RHS) Colour Chart. The color values were determined between 12 pm and 2 pm in October 1997 at the experimental research station CPRO-DLO, Wageningen, the Netherlands.

Origin: Selected crossing from a controlled hybridization program.

Parentage:

Female parent.—Unnamed seedling.

Male parent.—Unnamed seedling.

Classification:

Botanical.—A hybrid of the genus Lilium L.

Commercial.—Oriental Lily cv. ‘Genova’.

Plant:

Form.—A single stem carrying numerous, alternately arranged pedicels.

Height.—Medium for the mature plant, approximately 90-100 cm, depending on cultivation conditions.

Growth Habit.—Vigorous.

Stem.—Circular in transverse section; anthocyanin pigmentation absent; internodal length uniform.

Foliage:

Quantity.—Approximately 35-45, depending on cultivation conditions.

Size of leaf.—Medium, approximately 8½ cm long and 2½ cm wide, depending on cultivation conditions.

Shape of leaf.—Generally ovate.

Texture.—Hardly any pubescence.

Color.—Medium green.

Bulb:

Size.—16 to 18 cm in circumference.

Color.—White/pink.

Bud:

Form.—Tapering like a lancet.

Size.—Short, 6 cm in length, depending on cultivation conditions.

Opening.—Unfolds normally in standard cultivation conditions.

Color.—Light grayish-pink just before opening.

Tepals.—Folded, three visible.

Flower:

Blooming habit.—Raceme.

Size.—Medium to large, approximately 18 cm, depending on cultivation conditions.

Borne.—Erect along a longitudinal axis.

Shape.—Generally, hexagonal star in form and bowl-shaped in cross-section.

Tepalage:

Number of Tepals.—6.

Arrangement.—Three inner and three outer.

Color:

Upper Surface.—Red-purple, RHS 68B, white-yellow mid-vein and edges fading to white.

Lower Surface.—Red-purple, between RHS 68B and RHS 68C.

Spotting or Marking.—Orange spots are present on the inner side of the throat which is dark-orange in color.

Pedicel:

Length.—Medium, approximately 8 cm, depending on cultivation conditions.

Form.—Tapering like a lancet.

Texture.—Papillose.

Disease Resistance.—Medium.

Fragrance.—Present.

Lasting Quality.—Excellent.

Reproductive organs:

Stamens and anthers:

Arrangements.—Anthers form a generally circular pattern at their upper ends.

Number.—6.

Length.—Medium, depending on cultivation conditions.

Filaments Color.—Light-green.

Pollen color.—Reddish-brown.

Anther color.—Orange.

Pistils.—Yellowish-green.

Stigma.—Gray.

Ovaries.—Triangular in shape.

I claim:

1. A new and distinct cultivar of Lily plant named ‘Genova’, as illustrated and described herein.
